(19) United States
(12) Patent Application Publication
(10) Pub. No.: US 2007/0238680 A1
(43) Pub. Date: Oct. 11, 2007

(54) Substance and process for preventing a genetic defect from being passed onto offspring

(76) Inventors: Clayton A. Dehn, Austin, TX (US); Samuel L. Prien, Shallowater, TX (US)

Correspondence Address: JEFFREY M HERSH;ATTORNEY AT LAW, 9305 HAPPY TRAIL, AUSTIN, TX, 78754, US

(21) Appl. No.: 11/398,332

(22) Filed: Apr. 5, 2006

(51) Int. Cl. A61K 48/00

(52) U.S. Cl. 514/44

(57) ABSTRACT

A substance and process for preventing reproductive cells containing a genetic defect from being passed onto offspring by creating conditions, such as by adding a particular substance to extra-cellular fluid, that triggers or activates the particular disease associated with the genetic defect in defective reproductive cells, rendering those cells incapable of fertilization, implantation or embryonic development, but without significantly impairing healthy reproductive cells from producing offspring. The invention is particularly suited for channelopathies, and more particularly, for application with sperm from a Quarter Horse that is heterozygous for hyperkalemic periodic paralysis (HYPP). A solution of potassium chloride is added to a sperm extender to produce a potassium concentration of about 0.71 mg/ml. Sperm is introduced to the resulting modified sperm extender for at least five minutes, then is artificially inseminated into a mare. The high potassium concentration activates the disease solely within the sperm containing the defective gene, resulting in leakage of sodium-ion channels that under the increased potassium concentration floods the cytoplasm with positive ions, preventing repolarization of the cell membrane, immobilizing the defective sperm. The potassium concentration does not prevent repolarization in healthy sperm, which retain motility and are capable of fertilizing the egg.

### FIELD OF THE INVENTION

This invention relates generally to the field of reproductive technologies, and more particularly to a substance and process for immobilizing reproductive cells containing a defective gene, such as a channelopathy, preventing the impaired reproductive cells from resulting in offspring. An example of the present invention relates to the genetic defect of hyperkalemic periodic paralysis (HYPP) in horses.

### BACKGROUND OF THE INVENTION

Hyperkalemic periodic paralysis (HYPP) is an inherited disease that affects more than 100,000 descendents of a single blood line of Quarter Horses, that of IMPRESSIVE, #0767246. HYPP also affects some Standardbreds, and in extremely rare cases, humans ( 1/100,000). When manifested, HYPP results in uncontrolled muscle twitching and profound muscle weakness that in severe cases results in collapse or death. HYPP is a channelopathy, a category of conditions found in many species of mammals related to faulty ion channels in cell membranes. In humans, the most notable channelopathy is cystic fibrosis.

The HYPP genetic defect results in the substitution of the wrong amino acid on one critical location of the protein that forms the voltage-gated sodium-ion channels. The amino acid leucine erroneously replaces phenylalanine in the alpha subunit of the protein. The defect generally occurs through a rare random mutation that curiously always results in the identical wrong amino acid substitution. It spread throughout the Quarter Horse population because IMPRESSIVE—despite suffering from HYPP—was the top-winning, top-producing Quarter Horse of all time.

In a healthy muscle cell, the sodium and potassium concentrations are primarily maintained by voltage-regulated sodium-ion channels and voltage-regulated potassium-ion channels located within the cell membrane. The levels do not remain constant, but instead fluctuates over a concentration range in a repeating cycle about 100 times each second. The cycle begins when various factors cause the transmembrane potential—the voltage drop across the cell membrane between the cytoplasm and the extra-cellular fluid (ECF)—to depolarize to a threshold level of about −60 mV from its equilibrium level of about −70 mV.

When this threshold potential level is reached, the electrochemical gradient causes the voltage-regulated sodium-ion channels to activate, allowing sodium ions to enter the cytoplasm through the opened channel. This results in rapid depolarization. As the positively charged sodium ions flow into the cell, the charge within the cytoplasm increases until the polarity reverses and the transmembrane potential reaches about +30 mV.

At this level, the sodium-ion channels close and voltage-regulated potassium-ion channels are activated. This stops the flow of sodium ions into the cytoplasm, and opens the channel gates for the positively charged potassium ions to flow out of the cell. This reversal in the flow of positively charged ions causes the membrane to repolarize, returning to its equilibrium state of about −70 mV. The potassium-ion channels then close, and the sodium- and potassium-ion channels reinitialize, allowing the cycle to repeat.

The flow of ions is also regulated—though to a far less extent—by sodium-potassium pumps and passive-transport pores. The sodium-potassium pumps periodically activate to pump potassium ions into the cytoplasm, and to expel sodium ions out of the cell. This helps to counterbalance the flow of sodium and potassium ions from their respective channels. Furthermore, passive-transport pores in the cell membrane allow sodium and potassium ions, as well as other ions, to flow into and out of the cell, depending upon the concentration gradients of the particular ion. This ion flow could result in an increase or decrease in the charge within the cytoplasm.

However, the change to the transmembrane potential caused by sodium-potassium pumps and the pores are insignificant compared to the rapid electrochemical change caused by the opening and closing of the sodium- and potassium-ion channels. Consequently, these features to do qualitatively change the general depolarization-repolarization cycle described above. They instead maintain the concentrations of sodium and potassium ions within a certain range.

In a muscle cell containing the HYPP genetic defect, the sodium-ion channels fail to deactivate properly after the rapid depolarization phase. The channels fail to close completely, allowing sodium ions to leak into the cytoplasm during the repolarization phase. Thus, the sodium ion leakage at least partially offsets the decrease in the charge within the cytoplasm caused by the potassium-ion channel ejecting potassium ions out of the cell

In many such cases, the leakage is not significant enough to substantially affect the cycle. However, under some conditions, the increased charge caused by the leakage may prevent the transmembrane potential from returning to its equilibrium state. Alternately, the continual flow of sodium ions into the cytoplasm may exceed the rate at which the sodium-potassium pumps and passive-transport pores can eject sodium ions, resulting in excessive sodium concentrations beyond the tolerance of the muscle cell. In either case, the depolarization-repolarization cycle is broken, leading to impairment and sometimes death of the cell.

While HYPP is treatable, the American Quarter Horse Association (AQHA) has classified HYPP along with other genetic aberrations such as parrot mouth as genetically undesirable, and has established new rules that begin barring horses born with the genetic defect from registry. In 2007, foals that test positive for HYPP with both genes (homozygous) (H/H) will not be registered. (See Rule 205(c)(3).) And the AQHA is currently considering further amending the rules to bar registration of heterozygous (N/H) foals, possibly as early as 2010.

The registry rule changes provide great incentive for horse breeders to eradicate the defective HYPP genes, at least to ensure newborn foals have no more than one defective HYPP gene. Quarter Horses that are eligible for registry are often worth an order of magnitude more than those that are ineligible for registration. And the owner of an N/H stallion reported losing $40,000 in expected stud fees this breeding season because of the rule change.

For a Quarter Horse that is homozygous positive (H/H) for the disease, there is no chance for its offspring to be born without the defect, as the foal will definitely receive one of the defective genes from that parent. However, if the horse is heterozygous positive (N/H), there is an equal chance that its offspring would receive the healthy or defective gene from that parent. If both parents are N/H, there is a 75 percent chance that the offspring will be born with at least one defective gene, and a 25 percent change of a foal with two defective genes.

One potential solution would be to develop a test for the HYPP gene for the embryo after fertilization, and abort the embryo should it contain the genetic defect. Any such procedure, however, would be time consuming, costly, and place the mare at some risk.

A second avenue for potential solutions would be to prevent the sperm containing the defect from fertilizing the egg, to prevent the egg containing the defect from being fertilized, or to prevent a fertilized egg from implantation or development in the uterus; but to allow healthy sperm, eggs or embryos to be fertilized or implanted and developed, as the case may be. It would be highly advantageous to utilize any of these techniques to prevent a defective gene from being passed to the offspring, and after widespread use in the species, to eradicate the defective gene from the gene pool itself.

To date, no one has published any attempt to employ one of these solutions, let alone actually devise a particular technique that is successful.

### SUMMARY OF THE INVENTION

The present invention solves the above-mentioned problems by exploiting the very weakness caused by the genetic defect itself as manifested in the sperm, egg or embryo. The particular reproduction cell or cluster of cells is exposed to external conditions that trigger the particular channelopathy in reproductive cells containing the genetic defect, thereby immobilizing or killing those cells without detrimentally affecting healthy reproductive cells A triggering event may be accomplished in many ways, such as by changing the transmembrane potential, directly or indirectly changing the ion concentrations either within the cytoplasm or outside the cell, or by introducing an electrical current, electrochemical current or magnetism. By controlling the depolarization of the transmembrane potential, various ion-channel gates may be activated, opened, and closed such that cells possessing the defective gene cannot keep pace with the flow of ions or the change in charge across the cell membrane, immobilizing them or causing cellular death, whereas healthy cells are able to regulate their ion and charge balance, leaving them unscathed. Consequently, only healthy reproductive cells will be employed in fertilization, preventing the defective genes from being passed onto the offspring.

In the particular case of a stallion that is heterozygous for HYPP (N/H), the preferred technique for the triggering event is to increase the potassium concentration in the extra-cellular fluid (ECF) to about 2.3 times the potassium concentration within the sperm cell. Because almost all Quarter Horses are bred using artificial insemination, this is most readily accomplished by adding a sufficient quantity of potassium chloride (KCI) to the equine sperm extender to increase the potassium concentration about 70 times that of a standard skim-milk-based sperm extender. The preferred embodiment comprises 133.45 mg/ml of potassium chloride dissolved into 1.0 ml. of double-distilled water. This 1.0-ml potassium-chloride solution is added to 99 ml. of equine sperm extender, preferably ARS-CST equine sperm extender, which is manufactured by Animal Reproduction Systems, of Chino, Calif. The potassium chloride may be added to the sperm extender during the manufacture of the sperm extender, or as an additive by the end user prior to the sperm is added to the extender. In either case, the additive should be refrigerated prior to being added to the sperm extender.

The N/H stallion's sperm is then introduced into and kept in the potassium-fortified sperm extender for at least five minutes, and preferably for 15 minutes, occasionally swirling the mixture to ensure that each sperm cell is surrounded by the high concentration of potassium ions. This ensures that a triggering event, that is, an HYPP episode, is induced in substantially all genetically defective sperm, immobilizing or killing them, while leaving the healthy sperm unimpaired. The breeder then artificially inseminates the mare using the sperm in the KCl-fortified sperm extender using standard techniques, except that it is preferable to double the number of sperm cells used to inseminate the mare. Fertilization will occur only by one of the healthy cells, thereby preventing the stallion from passing the HYPP-defective gene to the foal. (There may be some error factor in which an occasional HYPP-positive sperm cell is not immobilized and fertilizes the egg—final test results are not complete at the time the Application for this patent is being filed.) After prolonged, widespread use of this process, HYPP will be substantially removed from the gene pool.

The same general technique may be adapted for use with other channelopathies, and with other genetic defects in which the physiology of reproductive cells carrying the bad gene differs from that of healthy reproductive cells.

Other objects and advantages of the present invention will be described or become apparent as the preferred embodiment is shown in further detail in the drawings, and as described in the discussion below.

### BRIEF DESCRIPTION OF THE DRAWINGS

The several features and advantages of the present invention will be better understood from a reading of the following detailed description in conjunction with the drawings, in which:

FIG. 1 shows a cross-sectional drawing of the cell membrane of a sperm in which the transmembrane potential is at equilibrium, or resting state, in connection with the present invention;

FIG. 2 shows a cross-sectional drawing of the cell in FIG. 1 in the depolarization state in which the sodium-ion channel is activated;

FIG. 3 shows a cross-sectional drawing of the cell in FIG. 1 wherein the cell is healthy and in the repolarization state in which the sodium-ion channel is closed and the potassium-ion channel is open;

FIG. 4 shows a cross-sectional drawing of the cell in FIG. 1 wherein the cell contains the HYPP genetic defect and in which the sodium-ion channel fails to close, thereby leaking sodium into the cell, and the potassium-ion channel is open;

FIG. 5 shows a graph of the contrast between the transmembrane potential (voltage) in a healthy sperm cell and an HYPP-defected sperm cell over the range of a depolarization-repolarization cycle when subjected to the potassium-chloride solution of the present invention;

FIG. 6 shows a graph of test results of the percentage of sperm cells in N/N, N/H and H/H samples that remain motile at varying concentrations of potassium mixed into the sperm extender; and

FIG. 7 shows a flow chart for the preferred process used to create the modified sperm extender of the present invention, and the method of using it.

### DETAILED DESCRIPTION OF THE DRAWINGS

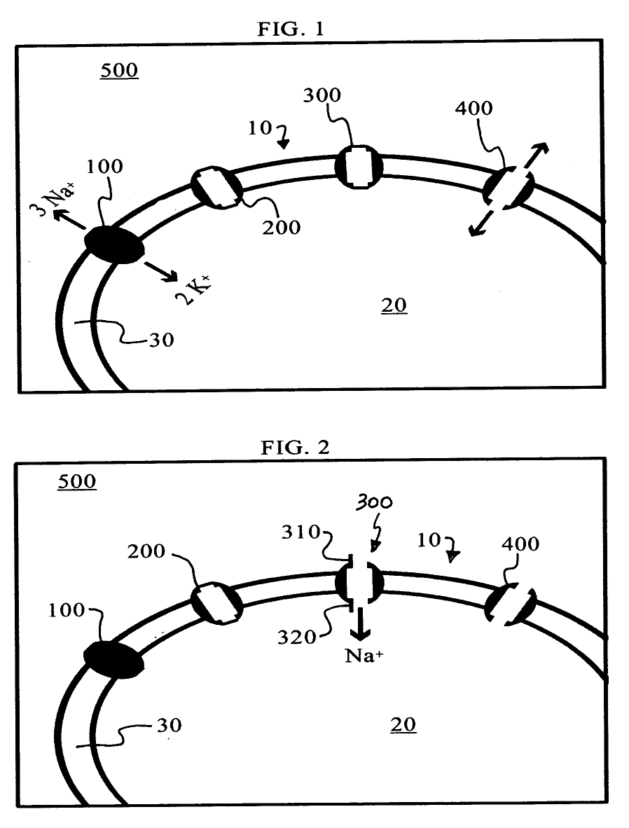

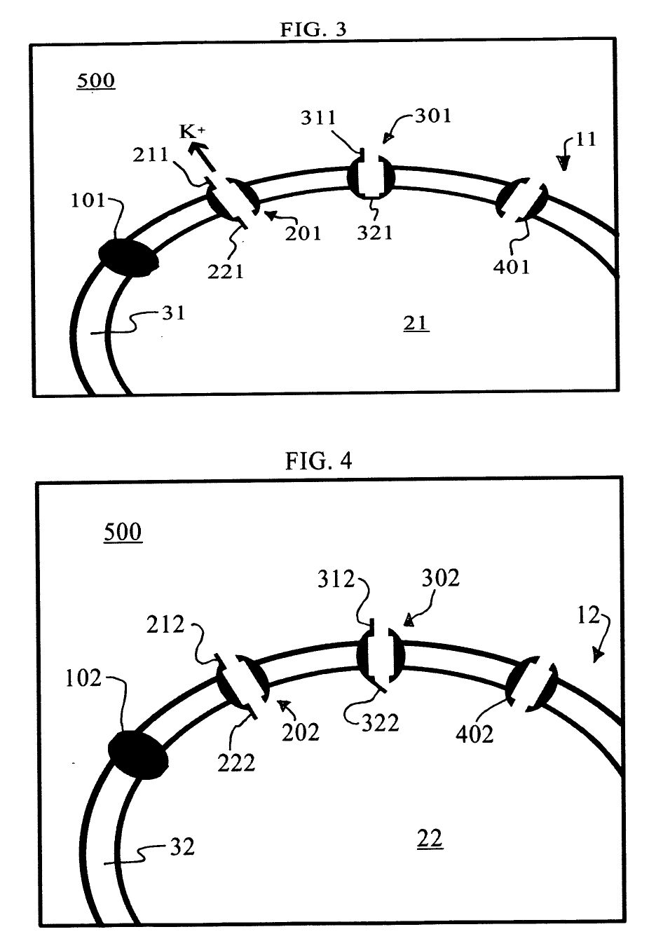

Identical reference numerals in the drawings denote the same elements throughout the various drawings. The reference numerals are further organized to aid the reader in comparing corresponding structures within sperm cells that are healthy with those that contain the HYPP-genetic defect. Where all the structures within the healthy and defective sperm cells function the same (i.e., in FIGS. 1-2), reference numerals end in “0”. Where any of the structures within the healthy and defective sperm cell function differently (i.e., in FIGS. 3-4), reference numerals end in “1” or “2”, depending upon whether the cell is healthy or defective, respectively. For example, in the first two phases of the depolarization-repolarization cycle where all cell components function identically in healthy and defective cells, the reference numerals for sodium-ion channel and its interior gate are “300” and “320,” respectively. But in the last phase, where there are some differences in the functioning of some cell components between the healthy and defective cells, reference numerals for the sodium-ion channel and its interior gate in the healthy cell are designated “301” and “321,” respectively, as shown in FIG. 3, and are designated in the defective cell as “302” and “322,” respectively, as shown in FIG. 4.

The present invention shall be described through the example of preventing the particular channelopathy in Quarter Horses known as hyperkalemic periodic paralysis (HYPP) from being passed from a stallion to its offspring. The present invention takes advantage of the fact that after meiosis, each particular sperm possesses only one of each chromosomal pair, and that gene affects the physiology of that particular sperm cell. Accordingly, sperm cells containing the HYPP defective gene suffer from the HYPP disease, whereas sperm cells containing healthy genes do not.

The crux of the present invention relates to subjecting the stallion's sperm to a triggering event, that is, conditions that spark the onset of the HYPP disorder in particular sperm carrying the defective gene. A sperm cell is essentially a highly modified muscle cell. Both share cytoskeletal filaments, which allow for contractions in the muscle cells and motility in sperm cells, movement of the sperm's tail or flagellum. Accordingly, the conditions that trigger an HYPP episode in muscle cells can be used to trigger an HYPP episode in a sperm cell that possesses the defective gene.

In the present invention, a triggering event must be sufficient to immobilize the defective sperm cells without damaging or immobilizing healthy sperm cells. In this way, only healthy sperm cells are able to fertilize the mare's egg, thereby preventing the stallion's HYPP gene from entering the genome type of the offspring. If the technique is consistently used throughout the line of affected Quarter Horses, the genetic defect would eventually be substantially eliminated from the gene pool. While the present invention is described in terms of HYPP in sperm cells only, the general technique is applicable and adaptable to other channelopathies in other species, including cystic fibrosis in humans.

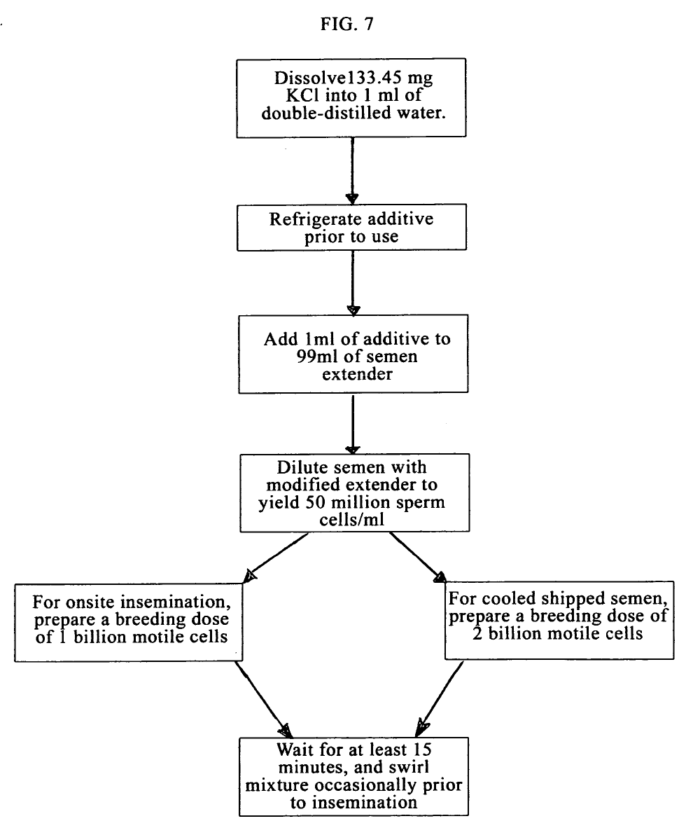

Turning now to the drawings, FIG. 1 shows sperm cell 10—which may be contain either the healthy or HYPP defective gene—surrounded by extra-cellular fluid (ECF) 500. FIG. 7 shows a flow chart for the process of making and using ECF 500. In the preferred embodiment, ECF 500 comprises ARS-CST equine sperm extender, manufactured by Animal Reproduction Systems, based in Chino, Calif. (although any standard skim-milk-based equine sperm extender may be used) mixed with sufficient quantities of potassium chloride to yield a potassium concentration that is about 70 times that of standard sperm extender. The potassium chloride may either be added to the sperm extender prior to its shipment and sale, or it may be sold separately as an additive—preferably in a double-distilled water solution sold in a separate vial—in which the customer or breeder would mix the additive into the sperm extender. The preferred proportional quantities are 133.45 mg/ml of potassium chloride (i.e., 70 mg/ml of potassium) mixed with 1.0 ml of water, which is then added to 99 ml. of sperm extender. The additive, or modified sperm extender, as the case may be, should be refrigerated prior to use.

The potassium chloride liquid is mixed with the sperm extender to form ECF 500 prior to introduction of the semen to ensure uniform potassium concentration in ECF 500 when exposed to the sperm cells. Semen should remain in ECF 500 for at least five minutes, and preferably for 15 minutes, occasionally swirling, to ensure immobilization of all HYPP sperm cells prior to artificial insemination. Standard handling procedures should be followed during the period prior to artificial insemination. Similarly, artificial insemination should proceed in accordance with standard practices, with the exception that the quantity of sperm cells per breeding dose should preferably be doubled. For on-site insemination, 1 billion motile cells should be used per breeding dose; for semen to be shipped for insemination, 2 billion motile cells should be used per breeding dose. This will compensate for any healthy sperm cells that may become immobilized under the high potassium concentrations, and ensure a high fertility rate.

Returning now to the drawings, the technique shall be described in connection with a single depolarization-repolarization cycle, shown in FIGS. 1-4, explaining the effect that the increased potassium chloride concentration has on both healthy and genetically defective HYPP sperm cells.

In FIGS. 1-2, sperm cell 10 comprises cell membrane 30, cytoplasm 20, sodium-potassium pump 100, potassium-ion channel 200, sodium-ion channel 300 and passive-transport pore 400, and is surrounded by ECF 500. While the drawings depict one of each of sodium-potassium pump 100, potassium-ion channel 200, sodium-ion channel 300 and passive transport pore 400, each cell 10 includes numerous of each of these pumps, channels and pores. (Those skilled in the art will appreciate and understand that while the following discussion will describe these cellular components in the singular, as if each cell only contains one of each pump, channel and pore, the discussion shall be properly interpreted to mean that there are hundreds of each, all operating in parallel.)

Sodium-potassium pump 100 helps regulate the concentration of potassium and sodium ions in cell 10 using active transport. As shown in FIG. 1, when activated, sodium-potassium pump 100 expels three sodium ions from cell 10 and pumps two potassium ions into cell 10 in each pumping cycle. Passive-transport pore 400 also regulates the concentration of potassium and sodium, as well as that of other ions, in cell, but passively, in accordance with the gradient pressure of each ion. That is, ions will naturally flow through cell membrane 10 in the direction from higher to lower concentration of the particular ion. Passive-transport pore 400 functions passively, and is always allowing ions to flow through it.

However, the rate at which sodium-potassium pump 100 and passive-transport pore 400 exchange ions into and out of cell 10 pales in comparison to the rate at which sodium-ion channel 200 and potassium-channel 300 exchange ions. Accordingly, when either of these channels is activated, they will control the net flow of ions and the concomitant change in the relative charge of cytoplasm 20 and ECF 500, known as the transmembrane potential of cell membrane 30. Accordingly, arrows depicting the flow of ions through sodium-potassium pump 100 and passive-transport pore 400 are shown only in FIG. 1, the phase where both potassium- and sodium-ion channels 200, 300 are inactive.

Sodium-ion channel 300 comprises two gates, exterior gate 310 and interior gate 320, which determine when sodium-ion channel 300 is activated. While the mechanism of these gates is more complicated than described here, it is sufficient to understand that sodium ions pass from ECF 500 into cytoplasm 20 when both exterior and interior gates 310, 320 are open, as shown in FIG. 2, but that sodium ions do not pass through sodium-ion channel 300 when either exterior gate 310 or interior gate 320 is closed, or both are closed, as shown in FIGS. 1 and 3 (note that the corresponding reference numerals in FIG. 3 are 301, 311 and 321). Exterior gate 310 and interior gate 320 are activated and inactivated—that is, open and close—in response to transmembrane potential levels across cell membrane 30, which is described in greater detail below.

Similarly, potassium-ion channel 200 comprises two gates, exterior gate 210 and interior gate 220, which determine when potassium-ion channel 200 is activated. Potassium ions pass from cytoplasm 20 to ECF 500 (note this is the opposite direction that sodium ions flow through sodium-ion channel 300) when exterior and interior gates 210, 220 are open (designated exterior and interior gates 211, 221 in FIG. 3, and exterior and interior gates 212, 222 in FIG. 4). Potassium ions do not pass through potassium-ion channel 200 when either exterior gate 210 or interior gate 220 is closed, or both are closed, as shown in FIGS. 1-2.

The HYPP sperm example of the present invention will now be described in light of a single depolarization-repolarization cycle of cell 10. FIG. 1 shows cell 10 in the resting, or inactive, state. The transmembrane potential is about −70 mV, meaning that the charge of ECF 500 is about 70 mV greater than the charge of cytoplasm 20. Note that while the potassium ion concentration of ECF 500 is about 70 times higher than the potassium concentration of standard sperm extenders, the charge is not significantly effected as the added potassium chloride presents equal amounts of negatively charged chloride ions as positively charged potassium ions.

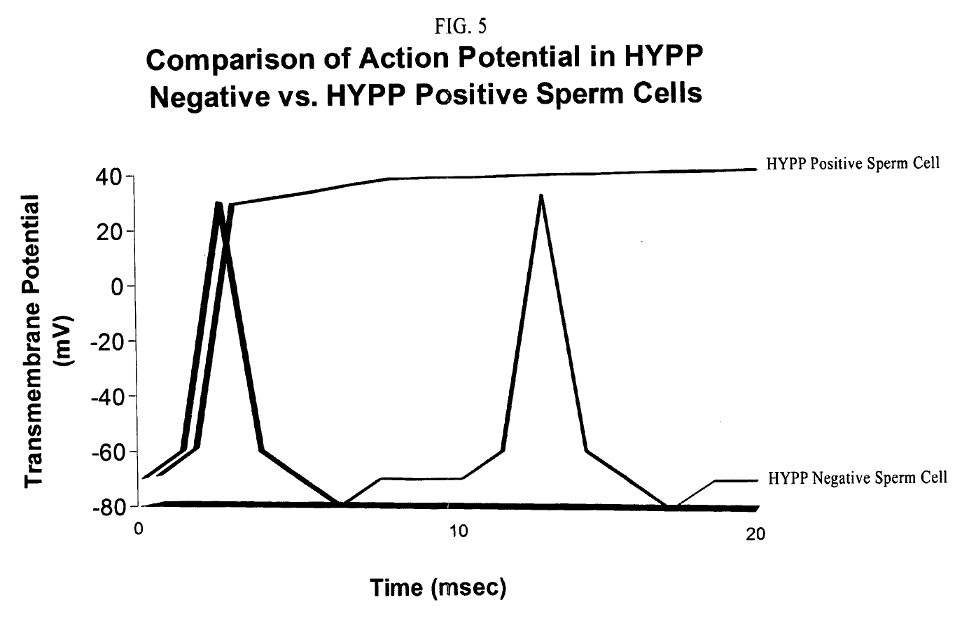

Various circumstances that are not significant in understanding the present invention cause the transmembrane potential across cell membrane 30 to depolarize. When the transmembrane potential increases to about −60 mV, sodium-ion channel 300 is activated, opening both exterior and interior gates 210, 220, as shown in FIG. 2. This allows sodium ions to flow into the cell at a relatively rapid rate, which speeds up the depolarization, as shown in FIG. 5, as positively charged sodium ions flow into cytoplasm 20 at a far faster rate than sodium-potassium pump 100 and passive-transport pore 400 can expel them. Consequently, the charge of cytoplasm 20 increases, causing the charge of cytoplasm 20 to become greater than the charge of ECF 500. The transmembrane potential across cell membrane 30 continues to increase until it reaches about +30 mV, at which point the depolarization phase of the cycle is complete.

At this stage, the physiology of cell 10 varies markedly depending upon whether cell 10 is healthy, or if it contains the defective HYPP gene. FIG. 3 shows a healthy cell 11 and the decrease in transmembrane potential across cell membrane 31, completing the depolarization-repolarization cycle. FIG. 4 shows an HYPP-defective cell 12 and the failure of its cell membrane 32 to repolarize, resulting in its immobilization. The difference in the transmembrane potential between healthy and defective sperm cells is shown the graph in FIG. 5.

In the healthy cell 11 in FIG. 3, when threshold transmembrane potential reaches about +30 mV, sodium-ion channel 301 deactivates, closing interior gate 321 and shutting off the flow of sodium ions into cytoplasm 21. Simultaneously, potassium-ion channel 201 activates, opening exterior and interior gates 211, 221, allowing potassium ions to flow out of cytoplasm 21 and into ECF 500. The combined result shifts the flow of positively charged ions across cell membrane 31, abruptly causing the transmembrane potential to decrease, thereby repolarizing cell 11, as shown in the graph in FIG. 5. When the transmember potential reaches about −70 mV, the resting or equilibrium potential for cell membrane 31, potassium-ion channel 201 is deactivated, and exterior and interior gates 211, 221 close, stopping the flow of potassium ions out of cytoplasm 21.

Potassium- and sodium-ion channels 201, 301 are then reinitialized, allowing the cycle to repeat, which it does in motile sperm cells about 100 times per second. This cycle is necessary and responsible for the propagation of the sperm cell, and is essential to enable the sperm cell to fertilize an egg.

In contrast, FIG. 4 shows the process in cell 12 that contains the HYPP defect. When the transmembrane potential reaches +30 mV, potassium-ion channel 202 activates the same as in the healthy cell 11, expelling positively charged potassium ions from cytoplasm 22, but sodium-ion channel 302 fails to deactivate properly. The genetic defect prevents interior gate 312 from closing completely, allowing sodium ions to continue to leak into cytoplasm 22. If the potassium concentration in ECF 500 was normal, such as if it consisted of standard sperm extender, the leakage would slow the repolarization process by offsetting slightly the flow of potassium ions out of cell 22, but would not necessarily prevent the repolarization process. However, with the high potassium concentration in ECF 500, the flow of potassium ions out of cytoplasm 22 through potassium-ion channel 202 is greatly reduced due to the high concentration gradient of potassium ions outside cell 22. This problem is exacerbated because the high concentration gradient of potassium ions outside cell 22 causes an increased flow of potassium ions into cytoplasm 22 through passive-transport pore 402. The net result of the ion flow is that the combined sodium and potassium concentration in cytoplasm 22 increases instead of decreasing, thereby causing the transmembrane potential to continue to increase, instead of decreasing. In other words, repolarization does not begin, depolarization continues, as shown in FIG. 5. Thus, the depolarization-repolarization cycle is broken, and the sperm becomes immobilized.

Because the high potassium concentration in ECF 500 causes sperm 12 having the HYPP defect to become immobilized while healthy sperm 11 retains motility, artificial insemination will result in fertilization of the egg by a healthy sperm. This prevents the offspring from obtaining the HYPP gene from its father.

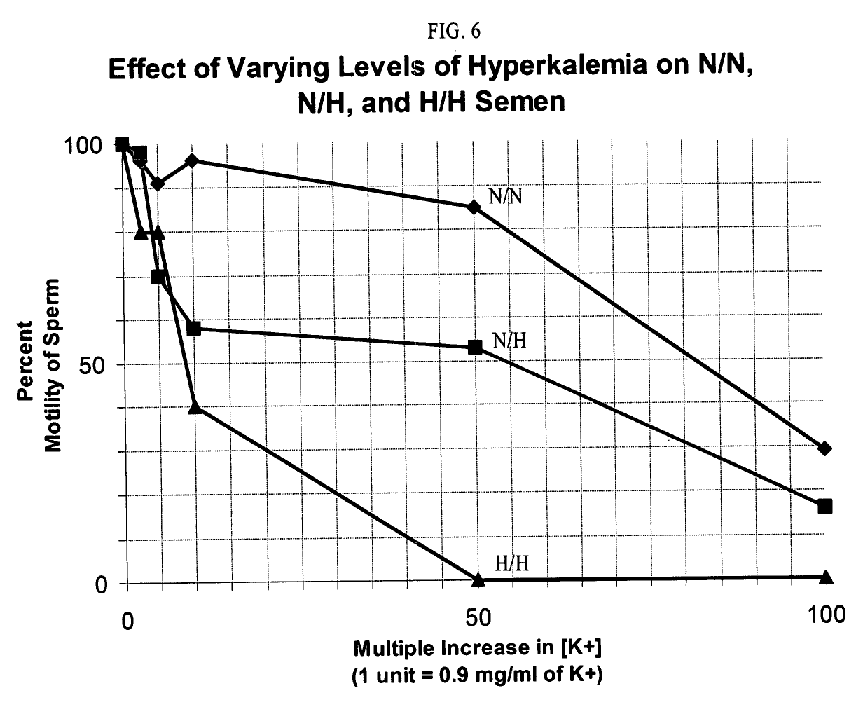

FIG. 6 shows a graph of the percent motility rate of sperm under various concentrations of potassium in ECF 500. Three curves are plotted—one for motility of N/N sperm, one for N/H sperm and one for H/H sperm. The x-axis shows the potassium concentration in terms of multiples of the potassium concentration of standard sperm extender, which is 0.9 mg/ml. In the tests, the sperm was kept in the particular sperm extender formula for about 24 hours. It was then diluted with standard sperm extender (with the low potassium concentration) at about an 8-to-1 ratio in a bullet tube, and its motility was measured using a computer-assisted semen analyzer. Note that the bold lines on the graph are linear extrapolations between the actual data points, which are shown in squares, triangles and diamonds, and are not accurate representations of the actual values between those data points.

As shown in the bottom-most curve in FIG. 6, as the potassium concentration increases, the motility of the H/H sperm sample drops to zero somewhere prior to 50 times the standard potassium concentration. Because all sperm in the H/H sample contain the defective sperm, we conclude that all defective sperm are immobilized at this concentration. More importantly, the sperm are permanently immobilized, as the test measured motility after the sperm sample was re-extended, restoring metabolic potassium levels. Furthermore, microscopic examination of the morphology of the sperm cells showed the tails were permanently curled around the cell body, indicating permanent immobility.

In contrast, the top-most curve shows the motility of the all-healthy N/N sperm sample decreases from about 100-percent motility at standard potassium levels to about 85-percent motility at 50 times standard potassium concentration. This indicates that healthy sperm cells are minimally affected by a 50-fold increase in potassium concentration. In fact, potassium concentrations of 100 times the normal potassium concentration left 29 percent of the sperm motile, whereas defective cells were completely and permanently impaired. Again, we expect that these sperm cells were temporarily immobilized under the high potassium concentration, and then resumed normal depolarization-repolarization cycling upon being diluted and measured in the computer-assisted semen analyzer.

The middle curve in FIG. 6 shows the more complicated sample of N/H sperm cells. One may expect that because this sample comprises half healthy cells, and half defected cells, that the motility percentage at a particular concentration would equal the average of the motility of the H/H and N/N samples. This, however, is not the case because the physiology of an HYPP-defective sperm cell from an N/H stallion differs from the physiology of an HYPP-defective sperm cell from an H/H stallion, even though the defective gene is identical in each case.

Prior to meiosis, both the healthy and defective genes are present in the primary spermatocyte cell, and each of the genes is active, creating both functional and faulty voltage-gated sodium-ion channels in the cell membrane. After this cell divides into two secondary spermatocyte cells, the cell membranes of the resulting two cells are made from the cell membrane from the primary spermatocyte cell, thereby each being comprised of about half functional and half faulty sodium-ion channels. However, the particular gene in each secondary spermantocyte cell—and the resulting two sperm cells into which they later divide—controls which sodium-ion channels are activated. In general, the gene in the HYPP-defective sperm cells activates the faulty sodium-ion channels, and the gene in the healthy sperm cells activates the functional sodium-ion channels. But while this is true in general, there is some cross-over activation. Various factors that will not be described in greater detail here result in a small number of healthy sodium-ion channels remaining active in defective cells, and a small number of faulty sodium-ion channels remaining active in healthy cells. We crudely estimate that less than 10 percent of healthy sodium-ion channels remain active in defective sperm from N/H stallions, and less than 10 percent of faulty sodium-ion channels remain active in healthy sperm from N/H stallions.

Accordingly, healthy sperm cells from an N/H stallion would be expected to be more sensitive to high potassium concentrations than healthy sperm cells from an N/N stallion—that is, we would expect a slightly lower motility percentage for healthy sperm cells from N/H stallions. And defective sperm cells from an N/H stallion would be expected to be less sensitive to high potassium concentrations than defective sperm cells from an N/N stallion—that is, we would expect a slightly higher motility percentage for healthy sperm cells from N/H stallions. In sum, higher potassium concentrations would be required to permanently immobilize all defective sperm from N/H stallions, which would further exacerbate the decrease in motility of healthy sperm from N/H stallions (as compared to healthy sperm from N/N stallions).

Consequently, the ideal potassium concentration would be expected to be higher than the minimal concentration at which all H/H sperm would become permanently immobilized, yet lower than the concentration that would otherwise appear to yield acceptable motility levels for healthy cells in N/N sperm sample. Accordingly, great care must be used in determining the appropriate concentration. (This dynamic would occur in employing this technique in other reproductive cells, and with other channelopathies.)

Turning now to the test data for the N/H sperm cell sample in FIG. 6, we observe that the motility percentage of the combined healthy and unhealthy sperm cells is about 53 percent at a potassium concentration of 50 times the normal concentration. If the healthy and defective sperm cells in this sample were physiologically the same as those from the N/N and H/H samples, then we would expect the motility percentage to be about 42.5 percent, the average of the other two samples, and certainly less than 50 percent. However, the test results are consistent with the cross-over dynamics described above.

Accordingly, to select the preferred potassium concentration, we extrapolate the curve so that the motility percentage is significantly less than 50 percent to compensate for the healthy sperm that become permanently immobilized. However, we did not desire a potassium concentration that resulted in any less than 60 percent motility for the N/N cell sample because of the risk of reduced fertility rate. The preferred potassium concentration was selected as 70 times the normal concentration, which yielded about a 38-percent motility rate for N/H sperm cells, and about a 60-percent motility rate for N/N sperm cells.

One anomaly that the inventors do not presently understand is that of several potassium-based salts that were tried, only potassium chloride yielded a suitable triggering event that impaired genetically defected sperm without also impairing healthy sperm. Potassium hydroxide (KOH) and potassium phosphate (K₂PO₄) failed to produce such a suitable outcome, even when the resulting potassium concentration in the extra-cellular fluid was in the same range as that in potassium chloride.

A similar process may be employed for theoretically and experimentally determining suitable conditions for a so-called triggering event in any channelopathy or similar type of genetic defect, and with respect to any type of reproductive cell or cell cluster. The triggering event should be sufficiently stressful as to ensure permanent impairment of the genetically defective reproductive cells or cell clusters to prevent or substantially reduce fertilization, implantation or embryonic development, without being sufficiently stressful as to impair the healthy reproductive cells.

Additional advantages and modifications will readily occur to those skilled in the art. Thus while the preferred embodiment of the present invention has been disclosed and described herein, the invention in its broader aspects is not limited to the specific details, methods and representative devices shown and described herein. It will be understood by those skilled in the art that various changes in form and detail may be made therein without departing from the spirit and scope of the general inventive concept as defined by the appended claims and their equivalents.

In particular, the present invention is described through the limited example of creating a triggering event that prevents sperm from a horse that contains the HYPP genetic defect from fertilizing the mare's egg by activating the disease of the genetic defect, thereby immobilizing defective sperm cells, without impairing healthy sperm cells. In this example, one suitable triggering event was experimentally demonstrated to involve adjusting the extra-cellular fluid by inclusion of one particular salt at one particular concentration. The present invention also would function with other reproductive cells or cell clusters, and with other channelopathies and genetic defects provided the disease or impairment caused by the particular genetic defect could be activated within the particular reproductive cell or cell cluster. If so, a suitable triggering event could be devised that provided sufficient stress to trigger the disease in reproductive cells containing the defective gene and disrupt fertilization, implantation or embryonic development, without applying too much stress that impairs healthy reproductive cells.

We claim:

1. A process for selectively preventing genetically impaired reproductive cells from producing offspring, while allowing healthy reproductive cells to produce offspring, comprising the steps of:
subjecting the reproductive cells to conditions that activate the genetic disease caused by the genetic defect within the genetically impaired reproductive cells in a manner that at least substantially reduces those genetically impaired cells from producing offspring, without significantly impairing the healthy reproductive cells from producing offspring; and
allowing the pregnancy to proceed.
